(19) United States
(12) Patent Application Publication
(10) Pub. No.: US 2024/0390558 A1
(43) Pub. Date: Nov. 28, 2024

(54) PLATE-DERIVED GROWTH FACTOR FORMULATION FOR ENHANCING BONE FUSION

(71) Applicant: BioMimetic Therapeutics, LLC, Franklin, TN (US)

(72) Inventors: Sharath Sundararaj, Franklin, TN (US); Robert K. Shockley, Franklin, TN (US); Conan S. Young, Franklin, TN (US); Carrie Browning, Franklin, TN (US)

(21) Appl. No.: 18/793,507

(22) Filed: Aug. 2, 2024

Related U.S. Application Data
(62) Division of application No. 16/820,219, filed on Mar. 16, 2020, now abandoned.
(60) Provisional application No. 62/818,104, filed on Mar. 14, 2019.

(51) Int. Cl. A61L 27/54; A61K 38/18; A61L 27/24; A61L 27/42

(52) U.S. Cl. CPC A61L 27/54; A61L 27/24; A61L 27/425; A61L 27/427; A61K 38/1858; A61L 2430/02

(57) ABSTRACT

The present disclosure provides improved compositions comprising a solution of a platelet-derived growth factor and a biocompatible matrix that are useful for bone fusion procedures, such as spine fusion procedures. The compositions advantageously comprise a the PDGF solution and biocompatible matrix in about a 2:1 volume to weight (mL:g) ratio such that the compositions can be readily applied to a site of bone fusion using a syringe or cannula. In particular embodiments, the composition is useful in conjunction with a spine fusion cage in a spine fusion procedure. The present disclosure also provides methods of performing fusion procedures, such as spine fusion, and kits for fusion procedures.

### CROSS-REFERENCE TO RELATED APPLICATIONS

This application is a divisional of U.S. application Ser. No. 16/820,219, filed on Mar. 16, 2020, which claims the benefit of priority to U.S. Provisional Application No. 62/818, 104, filed on Mar. 14, 2019, the contents of which are hereby incorporated by reference in their entirety.

### TECHNICAL FIELD

The present disclosure generally relates to compositions comprising a platelet-derived growth factor (PDGF) solution in a biocompatible matrix, and methods of their use in bone fusion procedures, such as spine fusion. The present disclosure further provides methods for preparing these compositions and kits for bone fusion.

### BACKGROUND OF THE INVENTION

Musculoskeletal problems are pervasive throughout the population in all age groups and in both sexes. Half of Americans will need services for fractures or bone fusions at some point in their lifetime according to a widely published article presented at the 2003 annual meeting of the American Academy of Orthopedic Surgeons (AAOS). Bone fusion is a common form of orthopedic surgery used to treat musculoskeletal problems associated with various joints of patients. Bone fusion involves the artificial induction of joint ossification between two bones. Approximately 100,000 lumbar spine fusion surgeries are performed annually in the U.S. in patients with lower back pain due to degenerative disc disease, deformity, unstable spine trauma, or spondylolisthesis. Spine fusion failure rates currently are as high as 30%. The use of various orthopedic devices such as pedicle screws, plates and osteobiologics has led to an improvement in fusion rates, but failed spinal surgeries still remain a serious problem. Spinal fusions in people and animals are performed to relieve back pain and associated neurologic signs including neck, arm, and leg pain, and generally require fusion of the pathologic spine segment and decompression of the spinal cord at the same level.

Bony fusion of adjacent vertebral bodies (VBs) of the spine is currently used as a solution to treat intervertebral disc disease. The diseased disc tissue is removed, the endplates of the adjacent vertebral bodies are debrided, and rigid fixation in the form of a cage to stabilize the two levels is implanted between the VBs. Healing progresses with the formation of new bone between the VBs throughout the cage hardware. In many cases, a bone graft of is used to ensure bony fusion across the cage gap and to stabilize the spine for the patient, alleviating pain and often returning the patient to some level of activity. In co-morbid patients, it may be challenging to achieve boney fusion across the gap between the VBs, and an osteoconductive and osteoinductive graft is used to enhance the bone healing response.

Both interbody and posterolateral spine fusions use a biological osteoinductive agent to generate the bone required for the spinal fusion. The current standard of care is to harvest bone from the iliac crest or other bony site to obtain an osteoinductive graft material. Accordingly, the patient must also undergo a procedure to harvest the graft, usually from the iliac crest, in addition to the spine fusion surgery itself. Harvesting bone graft from the iliac crest is associated with post-operative pain that can persist in up to 25% of patients. Other disadvantages associated with this treatment method are an increased risk of infection, availability and quality of bone autograft material, and the patient inconvenience that is caused due to donor site morbidity.

Accordingly, there is a need for improved bone graft compositions that are useful in bone fusion procedures. Furthermore, there is a need for a graft compositions that can be easily provided with a spine fusion cage during spine fusion procedures. Additionally, a compositions that can remain in the cage during and after a spine fusion procedure, thereby reducing movement of the graft material into the tissue outside of the cage so that the graft and any osteoconductive or osteoinductive agents remain localized at the fusion site. Finally, compositions that obviate the need for autograft harvesting would be useful. The present disclosure addresses these needs.

### BRIEF SUMMARY

The present disclosure provides in one embodiment a composition comprising a solution of platelet-derived growth factor (PDGF) disposed in a biocompatible matrix, the biocompatible matrix comprising a bone scaffolding material and a biocompatible binder, wherein the solution has a PDGF concentration ranging from about 0.05 mg/mL to about 5 mg/mL, and wherein the solution and the biocompatible matrix are present in the composition in a volume to mass ratio (mL:g) ranging from about 1:1 to about 2.5:1.

In some embodiments, the volume to mass ratio (mL/g) of the solution of PDGF to biocompatible matrix ranges from about 1.5:1 to about 2.5:1, while in other embodiments, the volume to mass ratio (mL/g) of the solution of PDGF to biocompatible matrix is about 2:1.

In some embodiments, the scaffolding material is chosen from porous calcium phosphate, calcium sulfate, allograft and combinations thereof.

In some embodiments, the porous calcium phosphate is selected from the group consisting of tricalcium phosphate, hydroxyapatite, poorly crystalline hydroxyapatite, amorphous calcium phosphate, calcium metaphosphate, dicalcium phosphate dihydrate, heptacalcium phosphate, calcium pyrophosphate dihydrate, calcium pyrophosphate, octacalcium phosphate and mixtures thereof, while in other embodiments, the calcium phosphate comprises β-tricalcium phosphate.

In still other embodiments, the bone scaffolding material comprises calcium phosphate and calcium sulfate.

In some embodiments, the PDGF is present in the solution at a concentration ranging from about 0.1 mg/ml to about 1.0 mg/ml, about 0.2 mg/ml to about 0.4 mg/ml, or about 0.3 mg/ml.

In some embodiments, the PDGF comprises PDGF-AA, PDGF-BB, PDGF-AB, PDGF-CC, PDGF-DD, or a mixture or a derivative thereof. In some embodiments, the PDGF comprises PDGF-BB, while in other embodiments, the PDGF consists of PDGF-BB. In some embodiments, the PDGF-BB comprises at least 65% intact PDGF-BB. In still other embodiments, the composition of any one of claims 12 to 13 wherein the PDGF-BB is recombinant human (rh) PDGF-BB.

In some embodiments, the solution comprises PDGF in a buffer, while in other embodiments, the solution consists of PDGF in a buffer. In some embodiments, the buffer comprises sodium acetate.

In some embodiments, the bone scaffolding material comprises particles in a range of about 50 microns to about 5000 microns in size, about 50 microns to about 5000 microns in size, about 100 microns to about 5000 microns in size, about 100 microns to about 5000 microns in size, about 100 microns to about 300 microns in size, about 100 microns to about 300 microns in size, or about 250 microns to about 1000 microns in size.

In some embodiments, the bone scaffolding material comprises porosity greater than about 25%, greater than about 40%, greater than about 50%, greater than about 80% or greater than about 90%.

In some embodiments, the bone scaffolding material comprises macroporosity.

In some embodiments, the bone scaffolding material has a porosity that facilitates cell migration into the matrix.

In some embodiments, the bone scaffolding material comprises interconnected pores.

In some embodiments, the bone scaffolding material is resorbable.

In some embodiments, the biocompatible binder comprises collagen.

In some embodiments, the biocompatible binder is present in the biocompatible matrix in an amount ranging from about 10 weight percent to about 40 weight percent, while in other embodiments, the biocompatible binder is present in the biocompatible matrix in an amount ranging from about 15 weight percent to about 35 weight percent, about 15 weight percent to about 25 weight percent, or about 20 weight percent.

In some embodiments, the biocompatible matrix consists of calcium phosphate and collagen.

In other embodiments, the present disclosure provides a method for fusing bone comprising administering the composition of any of the preceding embodiments to a desired site of bone fusion. In some embodiments, the site of bone fusion is in a joint. In some embodiments, the bone fusion is in a foot, toe, ankle, knee, hip, spine, rib, sternum, clavicle, joint, shoulder, scapula, elbow, wrist, hand or finger. In some embodiments, the bone fusion is a spine fusion.

In some embodiments, the method further comprising disposing an intravertebral spacer between vertebral bodies. In some embodiments, the composition is disposed in the vertebral spacer prior to inserting the vertebral spacer between the vertebral bodies. In other embodiments,

The method further comprising disposing the composition in the spacer after inserting the spacer between the vertebral bodies.

In some embodiments, the spine fusion procedure is an interbody fusion procedure, while in other embodiments, the spine fusion procedure is a lumbar fusion procedure.

The present disclosure additionally provides a kit for use in a bone fusion procedure comprising: a biocompatible matrix in a first container; a solution of PDGF in a second container, wherein the biocompatible matrix and solution of PDGF have a volume to mass ratio (mL:g) ranging from about 1:1 to about 2.5:1; and instructions for i) preparing a described in any of the aforementioned embodiments, and ii) administering the composition to a site of bone fusion.

### BRIEF DESCRIPTION OF THE DRAWINGS

FIGS. 1A-1F are photographic images depicting the preparation of an exemplary composition of the present disclosure.

FIG. 2 is a schematic depicting an exemplary process for mixing a biocompatible matrix with a solution of PDGF.

FIG. 3A depicts an exemplary spine fusion cage.

FIG. 3B depicts exemplary fixation hardware for use with a spine a fusion cage.

FIG. 3C depicts and exemplary spine fusion cage filled with a composition of the present disclosure.

FIGS. 4A-4C are graphs summarizing the results of biomechanical testing in an ovine model for spine fusion. Flexion extension range of motion (FIG. 4A), lateral bending range of motion (FIG. 4B), and axial rotation range of motion (FIG. 4C) was tested from sheep receiving either autograft, a composition of rhPDGF-BB with a collagen/β-TCP matrix, a collagen/β-TCP matrix without rhPDGF, or an empty cage. FIG. 4D is key to the graphs in FIGS. 4A-4C.

FIGS. 5A and 5B are graphs summarizing the bone volume fraction and bone density fraction, respectively, in an ovine model for spine fusion.

FIG. 6 is a photographic image of MicroCT scans of the mid-sagittal plane from sheep treated with autograft, a composition of rhPDGF-BB with a collagen/β-TCP matrix, a collagen/β-TCP matrix without rhPDGF, or an empty cage.

FIGS. 7A-7C are graphs summarizing the histomorphometric analysis of an ovine model for spine fusion. Mean bone percentage (FIG. 7A), mean soft tissue percentage (FIG. 7B) and mean empty space percentage (FIG. 7C) were analyzed.

FIG. 8 is a photograph image of histological sections of the mid-sagittal plane from sheep treated with autograft, a composition of rhPDGF-BB with a collagen/β-TCP matrix, a collagen/β-TCP matrix without rhPDGF, or an empty cage.

FIGS. 9 is a graph depicting serum cytokine levels in the high-dose rhPDGF-BB and autograft groups represented as fold change over time.

FIG. 10 is a graph depicting Mean volumes of hyperintense areas on T2-weighted MRI images over time. cytokine quantification at 4, 7, 10, and 21 days postoperatively in a rat paraspinal implant safety model.

FIG. 11 is a photographic image of representative T2-weighted axial MRI slices of the lumbar spine at L4-L5 disc space with overlays representing areas of inflammation.

FIGS. 12A and 12B are representative images of IF staining shown for Day 4 with β-TCP/Col 3.0mg of rhPDGF-BB for Ki67 (FIG. 12A) and von Willebrand Factor (vWF) (FIG. 12C).

FIGS. 13A and 13B are graphs depicting quantified IF staining for Ki67 (FIG. 13A) and vWF (FIG. 13B shown at days 4, 7, 10 and 21 post-operatively. FIG. 13C is a key to FIGS. 13A and 13B.

### DETAILED DESCRIPTION

The present disclosure provides compositions comprising a solution of PDGF in a biocompatible matrix and methods of using them for bone fusion procedures, particularly spine fusion procedures. When provided in a ratio of about 2:1 volume of PDGF solution: mass of biocompatible matrix (mL:g), the composition is a thick paste that can be applied through a cannula or needle to the site requiring treatment. In some embodiments, the biocompatible matrix and the solution comprising PDGF are present in the composition in a volume to mass ratio (mL:g) ranging from about 1:1 to about 2.5:1, about 1.5:1 to about 2.5:1, or about 2:1. In some embodiments, the composition is used in conjunction with a cage or intervertebral spacer. The composition may be deposited inside the lumen of the cage either prior to implantation of the cage between debrided vertebral bodies or after the cage has been placed between the vertebral bodies during a spine fusion surgical procedure.

Interbody fusion places a bone graft (e.g. a composition of the invention) between the vertebrae in the area usually occupied by an intervertebral disc. In preparation for the spinal fusion, the disc may be removed entirely. A device may be placed between the vertebrae to maintain spine alignment and disc height. The intervertebral device may be, for example, a spacer.

The intervertebral device may be made from, for example, plastic or titanium. The fusion then occurs between the endplates of the vertebrae. Types of interbody fusion include: anterior lumbar interbody fusion (ALIF), posterior lumbar interbody fusion (PLIF), and transforaminal lumbar interbody fusion (TLIF). In some embodiments, the fusion is augmented by a process called fixation, meaning the placement of metallic screws (pedicle screws often made from titanium), rods or plates, spacers, or cages to stabilize the vertebrae to facilitate bone fusion. During the fusion process, external bracing (orthotics) may be used.

Posterolateral fusion places the bone graft between the transverse processes in the back of the spine. These vertebrae may then be fixed in place with screws and/or wire through the pedicles of each vertebrae attaching to a metal rod on each side of the vertebrae.

### Definitions

As used herein, “promoting” or “facilitating” spinal fusion refers to a clinical intervention designed to desirably affect clinical progression of a spinal fusion procedure. Desirable effects of the clinical intervention include but are not limited to, for example, one or more of: increase in degree of bone density and/or acceleration of bone formation (e.g. acceleration of bone density) at the site of fusion, increase in degree of bony union or bone bridging and/or acceleration of bony union or bony bridging at the site of fusion, improvement in composition and/or structure of bone at bone fusion site (for example, closer resemblance to natural bone at the bone fusion site).

As used herein, the term “effective amount” refers to at least an amount effective, at dosages and for periods of time necessary, to achieve the desired therapeutic result. An effective amount can be provided in one or more administrations.

Reference to “about” a value or parameter herein also includes (and describes) embodiments that are directed to that value or parameter per se.

As used herein and in the appended claims, the singular forms “a,” “an,” and “the” include plural reference unless the context clearly indicates otherwise. For example, reference to a “PDGF homodimer” is a reference to one or multiple PDGF homodimers, and includes equivalents thereof known to those skilled in the art, and so forth.

It is understood that all aspects and embodiments of the invention described herein may include “comprising,” “consisting,” and “consisting essentially of” aspects and embodiments. It is to be understood that methods or compositions “consisting essentially of” the recited elements include only the specified steps or materials and those that do not materially affect the basic and novel characteristics of those methods and compositions.

“Bone scaffolding material” and “bone substituting agent” are used interchangeably in this disclosure.

“Volume to mass ratio” as used herein refers to ratio of the volume of PDGF in milliliters (mL) to the mass of biocompatible matrix in grams (g). For example, the volume to mass ratio can range from about 1:1 to about 2.5:1, about 1.5:1 to about 2.5:1, about 1.75:1 to about 2.25:1,or about 2:1. Exemplary embodiments may comprise, for example, about 2 mL of PDGF solution and about 1 g of biocompatible matrix, about 1.5 mL of PDGF and about 0.75 g of biocompatible matrix, or about 1 mL of PDGF solution and about 0.5 g of biocompatible matrix.

### PDGF Solutions

In one embodiment, a composition for spine fusion procedures comprises a solution comprising PDGF and a biocompatible matrix, wherein the solution is disposed or incorporated into the biocompatible matrix. In some embodiments, PDGF is present in the solution in a concentration ranging from about 0.01 mg/ml to about 10 mg/ml, from about 0.05 mg/ml to about 5 mg/ml, or from about 0.1 mg/ml to about 1.0 mg/ml. PDGF may be present in the solution at any concentration within these stated ranges, including the upper limit and lower limit of each range. In other embodiments, PDGF is present in the solution at any one of the following concentrations: about 0.05 mg/ml; about 0.1 mg/ml; about 0.15 mg/ml; about 0.2 mg/ml; about 0.25 mg/ml; about 0.3 mg/ml; about 0.35 mg/ml; about 0.4 mg/ml; about 0.45 mg/ml; about 0.5 mg/ml; about 0.55mg/ml; about 0.6 mg/ml; about 0.65 mg/ml; about 0.7 mg/ml; about 0.75 mg/ml; about 0.8 mg/ml; about 0.85 mg/ml; about 0.9 mg/ml; about 0.95 mg/ml; or about 1.0 mg/ml. It is to be understood that these concentrations are simply examples of particular embodiments, and that the concentration of PDGF may be within any of the concentration ranges stated above, including the upper limit and lower limit of each range.

Various amounts of PDGF may be used in the compositions of the present invention. Amounts of PDGF that are used, in some embodiments, include amounts in the following ranges: about 1 μg to about 50 mg, about 10 μg to about 25 mg, about 100 μg to about 10 mg, or about 250 μg to about 5 mg.

The concentration of PDGF or other growth factors in some embodiments of the present disclosure can be determined by using an enzyme-linked immunoassay as described in U.S. Pat. Nos. 6,221,625, 5,747,273, and 5,290,708, incorporated herein by reference, or any other assay known in the art for determining PDGF concentration. When provided herein, the molar concentration of PDGF is determined based on the molecular weight (MW) of PDGF dimer (e.g., PDGF-BB; MW about 25 kDa).

PDGF may comprise PDGF homodimers and/or heterodimers, including PDGF-AA, PDGF-BB, PDGF-AB, PDGF-CC, PDGF-DD, and mixtures and derivatives thereof. In some embodiments, PDGF comprises PDGF-BB. In another embodiment PDGF comprises a recombinant human (rh) PDGF, such as rhPDGF-BB.

PDGF, in some embodiments, can be obtained from natural sources. In other embodiments, PDGF can be produced by recombinant DNA techniques. In other embodiments, PDGF or fragments thereof may be produced using peptide synthesis techniques known to one of ordinary skill in the art, such as solid phase peptide synthesis. When obtained from natural sources, PDGF can be derived from biological fluids. Biological fluids, according to some embodiments, can comprise any treated or untreated fluid associated with living organisms including blood.

Biological fluids, in another embodiment, can also comprise blood components including platelet concentrate (PC), apheresed platelets, platelet-rich plasma (PRP), plasma, serum, fresh frozen plasma (FFP), and buffy coat (BC). Biological fluids, in a further embodiment, can comprise platelets separated from plasma and resuspended in a physiological fluid.

When PDGF is produced by recombinant DNA techniques, a DNA sequence encoding a single monomer (e.g., PDGF B-chain or A-chain), in some embodiments, can be inserted into cultured prokaryotic or eukaryotic cells for expression to subsequently produce the homodimer (e.g. PDGF-BB or PDGF-AA). In other embodiments, a PDGF heterodimer can be generated by inserting DNA sequences encoding for both monomeric units of the heterodimer into cultured prokaryotic or eukaryotic cells and allowing the translated monomeric units to be processed by the cells to produce the heterodimer (e.g. PDGF-AB). Commercially available GMP recombinant PDGF-BB can be obtained from Chiron Corporation (Emeryville, Calif.). Research grade rhPDGF-BB can be obtained from multiple sources including R&D Systems, Inc. (Minneapolis, Minn.), BD Biosciences (San Jose, Calif.), and Chemicon, International (Temecula, Calif.).

In some embodiments of the present invention, PDGF comprises PDGF fragments. In some embodiments rhPDGF-B comprises the following fragments: amino acid sequences 1-31, 1-32, 33-108, 33-109, and/or 1-108 of the entire B chain. The complete amino acid sequence (1-109) of the B chain of PDGF is provided in FIG. 15 of U.S. Pat. No. 5,516,896, the disclosure of which is hereby incorporated by reference in its entirety. It is to be understood that the rhPDGF-BB compositions of the present invention may comprise a combination of intact rhPDGF-B (1-109) and fragments thereof. Other fragments of PDGF may be employed such as those disclosed in U.S. Pat. No. 5,516,896. In accordance with one embodiment, the rhPDGF-BB comprises at least 65% of intact rhPDGF-B (1-109). In another embodiment, the rhPDGF-BB comprises at least 75%, 80%, 85%, 90%, 95%, or 99% of intact rhPDGF-B (1-109). Methods of producing PDGF are described in US Publication No. 20140308332, the contents of which are hereby incorporated by reference in their entirety.

In some embodiments, PDGF can be purified. Purified PDGF, as used herein, comprises compositions having greater than about 95% by weight PDGF prior to incorporation in solutions of the present invention. The solution may be any pharmaceutically acceptable solution. In other embodiments, the PDGF can be substantially purified. Substantially purified PDGF, as used herein, comprises compositions having about 5% to about 95% by weight PDGF prior to incorporation into solutions of the present invention. In some embodiments, substantially purified PDGF comprises compositions having about 65% to about 95% by weight PDGF prior to incorporation into solutions of the present invention. In other embodiments, substantially purified PDGF comprises compositions having about 70% to about 95%, about 75% to about 95%, about 80% to about 95%, about 85% to about 95%, or about 90% to about 95%, by weight PDGF, prior to incorporation into solutions of the present invention. Purified PDGF and substantially purified PDGF may be incorporated into scaffolds and binders.

In a further embodiment, PDGF can be partially purified. Partially purified PDGF, as used herein, comprises compositions having PDGF in the context of platelet rich plasma (PRP), fresh frozen plasma (FFP), or any other blood product that requires collection and separation to produce PDGF. Embodiments of the present invention contemplate that any of the PDGF isoforms provided herein, including homodimers and heterodimers, can be purified or partially purified. Compositions of the present invention containing PDGF mixtures may contain PDGF isoforms or PDGF fragments in partially purified proportions. Partially purified and purified PDGF, in some embodiments, can be prepared as described in U.S. patent application Ser. No. 11/159,533 (Publication No: 20060084602).

In some embodiments, solutions comprising PDGF are formed by solubilizing PDGF in aqueous media or in one or more buffers. Buffers suitable for use in PDGF solutions of the present invention can comprise, but are not limited to, carbonates, phosphates (e.g. phosphate buffered saline), histidine, acetates (e.g. sodium acetate), acidic buffers such as acetic acid and HCl, and organic buffers such as lysine, Tris buffers (e.g. tris (hydroxymethyl) aminoethane), N-2-hydroxyethylpiperazine-N′-2-ethanesulfonic acid (HEPES), and 3-(N-morpholino) propanesulfonic acid (MOPS). Buffers can be selected based on biocompatibility with PDGF and the buffer's ability to impede undesirable protein modification. Buffers can additionally be selected based on compatibility with host tissues. In some embodiments, sodium acetate buffer is used. The buffers can be employed at different molarities, for example, about 0.1 mM to about 100 mM, about 1 mM to about 50 mM, about 5 mM to about 40 mM, about 10 mM to about 30 mM, or about 15 mM to about 25 mM, or any molarity within these ranges. In some embodiments, an acetate buffer is employed at a molarity of about 20 mM.

In another embodiment, solutions comprising PDGF are formed by solubilizing lyophilized PDGF in water, wherein prior to solubilization the PDGF is lyophilized from an appropriate buffer.

Solutions comprising PDGF, according to embodiments of the present invention, can have a pH ranging from about 3.0 to about 8.0. In some embodiments, a solution comprising PDGF has a pH ranging from about 5.0 to about 8.0, from about 5.5 to about 7.0, or from about 5.5 to about 6.5, or any value within these ranges. The pH of solutions comprising PDGF, in same embodiments, can be compatible with the prolonged stability and efficacy of PDGF or any other desired biologically active agent. PDGF may be more stable in an acidic environment. Therefore, in accordance with one embodiment, the present invention comprises an acidic storage formulation of a PDGF solution. In accordance with this embodiment, the PDGF solution preferably has a pH from about 3.0 to about 7.0 or from about 4.0 to about 6.0. The biological activity of PDGF, however, can be optimized in a solution having a neutral pH range. Therefore, in a further embodiment, the present invention comprises a neutral pH formulation of a PDGF solution. In accordance with this embodiment, the PDGF solution has a pH from about 5.0 to about 8.0, from about 5.5 to about 7.0, or from about 5.5 to about 6.5. In accordance with a method of the present invention, an acidic PDGF solution is reformulated to a neutral pH composition. In accordance with a preferred embodiment of the present invention, the PDGF utilized in the solutions is rh-PDGF-BB. In a further embodiment, the pH of the PDGF containing solution can be altered to optimize the binding kinetics of PDGF to a biocompatible matrix.

The pH of solutions comprising PDGF, in some embodiments, can be controlled by the buffers recited herein. Various proteins demonstrate different pH ranges in which they are stable. Protein stabilities are primarily reflected by isoelectric points and charges on the proteins. The pH range can affect the conformational structure of a protein and the susceptibility of a protein to proteolytic degradation, hydrolysis, oxidation, and other processes that can result in modification to the structure and/or biological activity of the protein.

In some embodiments, solutions comprising PDGF can further comprise additional components, such as other biologically active agents. In other embodiments, solutions comprising PDGF can further comprise cell culture media, other stabilizing proteins such as albumin, antibacterial agents, protease inhibitors [e.g., ethylenediaminetetraacetic acid (EDTA), ethylene glycol-bis (beta-aminoethylether)-N,N,N′,N′-tetraacetic acid (EGTA), aprotinin, .epsilon.-aminocaproic acid (EACA), etc.] and/or other growth factors such as fibroblast growth factors (FGFs), epidermal growth factors (EGFs), transforming growth factors (TGFs), keratinocyte growth factors (KGFs), insulin-like growth factors (IGFs), bone morphogenetic proteins (BMPs), or other PDGFs including compositions of PDGF-AA, PDGF-BB, PDGF-AB, PDGF-CC and/or PDGF-DD.

### Biocompatible Matrix

The compositions comprise a biocompatible matrix. In some embodiments, the biocompatible matrix comprises one or more bone scaffolding and one or more biocompatible binders.

### Bone Scaffolding Material

A biocompatible matrix, according to some embodiments of the present invention, comprises a bone scaffolding material. It is to be understood that the terms bone scaffolding material and bone substituting agent are used interchangeably in this disclosure. The bone scaffolding material provides a framework or scaffold for new bone and tissue growth to occur. A bone substituting agent is one that can be used to permanently or temporarily replace bone. Following implantation, the bone substituting agent can be retained by the body or it can be resorbed by the body and replaced with bone. Exemplary bone substituting agents include, e.g., a calcium phosphate (e.g., tricalcium phosphate, such as β-tricalcium phosphate (β-TCP), hydroxyapatite, poorly crystalline hydroxyapatite, amorphous calcium phosphate, calcium metaphosphate, dicalcium phosphate dihydrate, heptacalcium phosphate, calcium pyrophosphate dihydrate, calcium pyrophosphate, and octacalcium phosphate), calcium sulfate, and allograft (e.g. mineralized bone, mineralized deproteinized xenograft, or demineralized bone (e.g., demineralized freeze-dried cortical or cancellous bone), and any combination thereof.

A bone scaffolding material, in some embodiments, comprises at least one calcium phosphate. In some embodiments, calcium phosphate comprises β-TCP. In other embodiments, a bone scaffolding material comprises a plurality of calcium phosphates. Calcium phosphates suitable for use as a bone scaffolding material, in some embodiments of the present invention, have a calcium to phosphorus atomic ratio ranging from 0.5 to 2.0. In some embodiments, a bone scaffolding material comprises allograft.

In some embodiments, biocompatible matrices may include calcium phosphate particles with or without biocompatible binders or bone allograft such as demineralized freeze dried bone allograft (DFDBA) or particulate demineralized bone matrix (DBM). In another embodiment, biocompatible matrices may include bone allograft such as DFDBA or DBM. In an embodiment, the biocompatible matrix is bioresorbable. In some embodiment, a biocompatible matrix comprises an allograft such as DFDBA or particulate DBM.

Non-limiting examples of calcium phosphates suitable for use as bone scaffolding materials comprise amorphous calcium phosphate, monocalcium phosphate monohydrate (MCPM), monocalcium phosphate anhydrous (MCPA), dicalcium phosphate dihydrate (DCPD), dicalcium phosphate anhydrous (DCPA), octacalcium phosphate (OCP), α-tricalcium phosphate, β-TCP, hydroxyapatite (OHAp), poorly crystalline hydroxapatite, tetracalcium phosphate (TTCP), heptacalcium decaphosphate, calcium metaphosphate, calcium pyrophosphate dihydrate, calcium pyrophosphate, carbonated calcium phosphate, or any mixture thereof.

In another embodiment, the bone substituting agent has a porous composition. Porosity is a desirable characteristic as it facilitates cell migration and infiltration into the implant material so that the infiltrating cells can secrete extracellular bone matrix. Porosity also provides access for vascularization. Porosity also provides a high surface area for enhanced resorption and release of active substances, as well as increased cell-matrix interaction. The composition can be provided in a shape suitable for implantation (e.g., a sphere, a cylinder, or a block) or it can be sized and shaped prior to use. In a preferred embodiment, the bone substituting agent is a calcium phosphate (e.g., β-TCP). Porous bone scaffolding materials, according to some embodiments, can comprise pores having diameters ranging from about 1 μm to about 1 mm. In some embodiments, a bone scaffolding material comprises macropores having diameters ranging from about 100 μm to about 1 mm. In another embodiment, a bone scaffolding material comprises mesopores having diameters ranging from about 10 μmm to about 100 μm. In a further embodiment, a bone scaffolding material comprises micropores having diameters less than about 10 μm. Embodiments of the present invention contemplate bone scaffolding materials comprising macropores, mesopores, and micropores or any combination thereof. In some embodiments, the bone scaffolding material comprises interconnected pores. In some embodiments, the bone scaffolding material comprises non-interconnected pores. In some embodiments, the bone scaffolding material comprises interconnected and non-interconnected pores.

A porous bone scaffolding material, in some embodiments, has a porosity greater than about 25% or greater than about 40%. In another embodiment, a porous bone scaffolding material has a porosity greater than about 50%, greater than about 60%, greater than about 65%, greater than about 70%, greater than about 80%, or greater than about 85%. In a further embodiment, a porous bone scaffolding material has a porosity greater than about 90%. In some embodiments, a porous bone scaffolding material comprises a porosity that facilitates cell migration into the scaffolding material.

In some embodiments, a bone scaffolding material comprises a plurality of particles. A bone scaffolding material, for example, can comprise a plurality of calcium phosphate particles. Particles of a bone scaffolding material, in some embodiments, can individually demonstrate any of the pore diameters and porosities provided herein for the bone scaffolding. In other embodiments, particles of a bone scaffolding material can form an association to produce a matrix having any of the pore diameters or porosities provided herein for the bone scaffolding material.

Bone scaffolding particles may be mm, μm or submicron (nm) in size. Bone scaffolding particles, in some embodiments, have an average diameter ranging from about 1 μm to about 5 mm. In other embodiments, particles have an average diameter ranging from about 1 mm to about 2 mm, from about 1 mm to about 3 mm, or from about 250 μm to about 750 μm. Bone scaffolding particles, in another embodiment, have an average diameter ranging from about 100 μm to about 300 μm. In a further embodiment, the particles have an average diameter ranging from about 75 μm to about 300 μm. In additional embodiments, bone scaffolding particles have an average diameter less than about 25 μm, less than about 1 μm and, in some cases, less than about 1 mm. In some embodiments, a bone scaffolding particles have an average diameter ranging from about 100 μm to about 5 mm or from about 100 μm to about 3 mm. In other embodiments, bone scaffolding particles have an average diameter ranging from about 250 μm to about 2 mm, from about 250 μm to about 1 mm, from about 200 μm to about 3 mm. Particles may also be in the range of about 1 nm to about 1000 nm, less than about 500 nm or less than about 250 nm.

Bone scaffolding particles, in some embodiments, have a diameter ranging from about 1 μm to about 5 mm. In other embodiments, particles have a diameter ranging from about 1 mm to about 2 mm, from about 1 mm to about 3 mm, or from about 250 μm to about 750 μm. Bone scaffolding particles, in another embodiment, have a diameter ranging from about 100 μm to about 300 μm. In a further embodiment, the particles have a diameter ranging from about 75 μm to about 300 μm. In additional embodiments, bone scaffolding particles have a diameter less than about 25 μm, less than about 1 μm and, in some cases, less than about 1 mm. In some embodiments, a bone scaffolding particles have a diameter ranging from about 100 μm to about 5 mm or from about 100 μm to about 3 mm. In other embodiments, bone scaffolding particles have a diameter ranging from about 250 μm to about 2 mm, from about 250 μm to about 1 mm, from about 200 μm to about 3 mm. Particles may also be in the range of about 1 nm to about 1000 nm, less than about 500 nm or less than about 250 nm.

In some embodiments, bone scaffolding materials are moldable, extrudable, and/or injectable. Moldable, extrudable, and/or injectable bone scaffolding materials can facilitate efficient placement of compositions of the present disclosure in and around target sites in bone and between bones at sites of desired bone fusion during spine fusion procedures. In some embodiments, moldable bone scaffolding materials can be applied to sites of bone fusion with a spatula or equivalent device. In some embodiments, bone scaffolding materials are flowable. Flowable bone scaffolding materials, in some embodiments, can be applied to sites of bone fusion through a syringe with a needle or cannula. In some embodiments, bone scaffolding materials harden in vivo.

In some embodiments, bone scaffolding materials are bioresorbable. A bone scaffolding material, in some embodiments, can be at least 30%, 40%, 50%, 60%, 70%, 75% or 90% resorbed within one year subsequent to in vivo implantation. In another embodiment, a bone scaffolding material can be resorbed at least 5%, 10%, 20%, 30%, 40%, 50%, 60%, 70%, 75% or 90% within 1, 3, 6, 9, 12, or 18 months of in vivo implantation. Bioresorbability will be dependent on: (1) the nature of the matrix material (i.e., its chemical make-up, physical structure and size); (2) the location within the body in which the matrix is placed; (3) the amount of matrix material that is used; (4) the metabolic state of the patient (diabetic/non-diabetic, osteoporotic, smoker, old age, steroid use, etc.); (5) the extent and/or type of injury treated; and (6) the use of other materials in addition to the matrix such as other bone anabolic, catabolic and anti-catabolic factors.

### Biocompatible Binder

Biocompatible binders, according to some embodiments, can comprise materials operable to promote cohesion between combined substances. A biocompatible binder, for example, can promote adhesion between particles of a bone scaffolding material in the formation of a biocompatible matrix.

Biocompatible binders, in some embodiments, can comprise collagen, polysaccharides, nucleic acids, carbohydrates, proteins, polypeptides, synthetic polymers, poly (α-hydroxy acids), poly (lactones), poly (amino acids), poly (anhydrides), polyurethanes, poly (orthoesters), poly (anhydride-co-imides), poly (orthocarbonates), poly (α-hydroxy alkanoates), poly (dioxanones), poly (phosphoesters), polylactic acid, poly (L-lactide) (PLLA), poly (D,L-lactide) (PDLLA), polyglycolide (PGA), poly (lactide-co-glycolide (PLGA), poly (L-lactide-co-D,L-lactide), poly (D,L-lactide-co-trimethylene carbonate), polyglycolic acid, polyhydroxybutyrate (PHB), poly (.epsilon.-caprolactone), poly (δ-valerolactone), poly (γ-butyrolactone), poly (caprolactone), polyacrylic acid, polycarboxylic acid, poly (allylamine hydrochloride), poly (diallyldimethylammonium chloride), poly (ethyleneimine), polypropylene fumarate, polyvinyl alcohol, polyvinylpyrrolidone, polyethylene, polymethylmethacrylate, carbon fibers, poly (ethylene glycol), poly (ethylene oxide), poly (vinyl alcohol), poly (vinylpyrrolidone), poly (ethyloxazoline), poly (ethylene oxide)-co-poly (propylene oxide) block copolymers, poly (ethylene terephthalate) polyamide, and copolymers and mixtures thereof.

Biocompatible binders, in other embodiments, can comprise alginic acid, arabic gum, guar gum, xantham gum, gelatin, chitin, chitosan, chitosan acetate, chitosan lactate, chondroitin sulfate, lecithin, N,O-carboxymethyl chitosan, phosphatidylcholine derivatives, a dextran (e.g., .alpha.-cyclodextrin, .beta.-cyclodextrin, .gamma.-cyclodextrin, or sodium dextran sulfate), fibrin glue, lecithin, glycerol, hyaluronic acid, sodium hyaluronate, a cellulose (e.g., methylcellulose, carboxymethylcellulose, hydroxypropyl methylcellulose, or hydroxyethyl cellulose), a glucosamine, a proteoglycan, a starch (e.g., hydroxyethyl starch or starch soluble), lactic acid, a pluronic acid, sodium glycerophosphate, glycogen, a keratin, silk, and derivatives and mixtures thereof.

In some embodiments, the binder comprises collagen. In some embodiments, the collagen comprises Type I collagen. In some embodiments, the collagen comprises bovine Type I collagen. In some embodiments, a biocompatible binder comprises hyaluronic acid.

In some embodiments, a biocompatible binder is water-soluble. A water-soluble binder can dissolve from the biocompatible matrix shortly after its implantation, thereby introducing macroporosity into the biocompatible matrix. Macroporosity, as discussed herein, can increase the osteoconductivity of the implant material by enhancing the access and, consequently, the remodeling activity of the osteoclasts and osteoblasts at the implant site.

In some embodiments, a biocompatible binder can be present in a biocompatible matrix in an amount ranging from about 1 weight percent to about 70 weight percent, about 5 weight percent to about 50 weight percent, about 10 weight percent to about 40 weight percent, about 15 weight percent to about 35 weight percent, or about 15 weight percent to about 25 weight percent of the biocompatible matrix. In a further embodiment, a biocompatible binder can be present in an amount of about 20 weight percent of the biocompatible matrix.

A biocompatible matrix comprising a bone scaffolding material and a biocompatible binder, according to some embodiments, can be flowable, moldable, and/or extrudable. In such embodiments, a biocompatible matrix can be in the form of a paste. A biocompatible matrix in the form of a paste, in some embodiments, can comprise particles of a bone scaffolding material adhered to one another by a biocompatible binder.

A biocompatible matrix in paste or putty form can be molded into the desired implant shape or can be molded to the contours of the implantation site. In some embodiments, a biocompatible matrix in paste or putty form can be injected into an implantation site with a syringe or cannula.

In some embodiments, a biocompatible matrix in paste or putty form does not harden and retains a flowable and moldable form subsequent to implantation. In other embodiments, a paste or putty can harden subsequent to implantation, thereby reducing matrix flowability and moldability.

A biocompatible matrix comprising a bone scaffolding material and a biocompatible binder, in some embodiments, can also be provided in a predetermined shape including a block, sphere, or cylinder or any desired shape, for example a shape defined by a mold or a site of application.

A biocompatible matrix comprising a bone scaffolding material and a biocompatible binder, in some embodiments, is bioresorbable as described above. A biocompatible matrix, in such embodiments, can be resorbed within one year of in vivo implantation. In another embodiment, a biocompatible matrix comprising a bone scaffolding material and a biocompatible binder can be resorbed within 1, 3, 6, or 9 months of in vivo implantation. Bioresorbability will be dependent on: (1) the nature of the matrix material (i.e., its chemical makeup, physical structure and size); (2) the location within the body in which the matrix is placed; (3) the amount of matrix material that is used; (4) the metabolic state of the patient (diabetic/non-diabetic, osteoporotic, smoker, old age, steroid use, etc.); (5) the extent and/or type of injury treated; and (6) the use of other materials in addition to the matrix such as other bone anabolic, catabolic and anti-catabolic factors.

While the following describes particular embodiments with reference to a bone scaffolding material comprising β-TCP and a biocompatible binder comprising collagen, it is to be understood that other embodiments of the invention may be produced by substituting other bone scaffolding material(s) (e.g. another calcium phosphate, calcium sulfate, or allograft) for the β-TCP, and/or by substituting other binder(s) for the collagen.

### Bone Scaffolding Material Comprising β-TCP

In some embodiments, a bone scaffolding material for use as a biocompatible matrix can comprise β-TCP. β-TCP, according to some embodiments, can comprise a porous structure having multidirectional and interconnected pores of varying diameters. In some embodiments, β-TCP comprises a plurality of pockets and non-interconnected pores of various diameters in addition to the interconnected pores. The porous structure of β-TCP, in some embodiments, comprises macropores having diameters ranging from about 100 μm to about 1 mm, mesopores having diameters ranging from about 10 μm to about 100 μm, and micropores having diameters less than about 10 μm. Macropores and micropores of the β-TCP can facilitate osteoinduction and osteoconduction while macropores, mesopores and micropores can permit fluid communication and nutrient transport to support bone regrowth throughout the β-TCP biocompatible matrix.

In comprising a porous structure, β-TCP, in some embodiments, can have a porosity greater than 25% or greater than about 40%. In other embodiments, β-TCP can have a porosity greater than 50%, greater than about 60%, greater than about 65%, greater than about 70%, greater than about 75%, greater than about 80%, or greater than about 85%. In a further embodiment, β-TCP can have a porosity greater than about 90%. In some embodiments, (β-TCP can have a porosity that facilitates cell migration into the β-TCP.

In some embodiments, a bone scaffolding material comprises β-TCP particles. β-TCP particles, in some embodiments, can individually demonstrate any of the pore diameters and porosities provided herein for β-TCP. In other embodiments, β-TCP particles of a bone scaffolding material can form an association to produce a matrix having any of the pore diameters or porosities provided herein for the bone scaffolding material. Porosity may facilitate cell migration and infiltration into the matrix for subsequent bone formation. β-TCP particles, in some embodiments, have an average diameter ranging from about 1 μm to about 5 mm. In other embodiments, β-TCP particles have an average diameter ranging from about 1 mm to about 2 mm, from about 1 mm to about 3 mm, from about 250 μm to about 750 μm, from about 250 μm to about 1 mm, from about 250 μm to about 2 mm, or from about 200 μm to about 3 mm. In another embodiment, β-TCP particles have an average diameter ranging from about 100 μm to about 300 μm. In a further embodiment, β-TCP particles have an average diameter ranging from about 75 μm to about 300 μm. In additional embodiments, β-TCP particles have an average diameter less than about 25 μm, average diameter less than about 1 μm, or less than about 1 mm. In some embodiments, β-TCP particles have an average diameter ranging from about 100 μm to about 5 mm or from about 100 μm to about 3 mm.

A biocompatible matrix comprising β-TCP particles, in some embodiments, can be provided in a shape suitable for implantation (e.g., a sphere, a cylinder, or a block). In other embodiments, a β-TCP bone scaffolding material can be moldable, extrudable, and/or injectable thereby facilitating placement of the matrix in and around target sites of desired bone fusion during spine fusion procedures. Flowable matrices may be applied through syringes, tubes, or spatulas or equivalent devices. Flowable β-TCP bone scaffolding materials, in some embodiments, can be applied to sites of bone fusion through a syringe and needle or cannula. In some embodiments, β-TCP bone scaffolding materials harden in vivo.

A β-TCP bone scaffolding material, according to some embodiments, is bioresorbable. In some embodiments, a β-TCP bone scaffolding material can be at least 30%, 40%, 50%, 60%, 65%, 70%, 75%, 80%, or 85% resorbed one year subsequent to in vivo implantation. In another embodiment, a β-TCP bone scaffolding material can be greater than about 90% resorbed one year subsequent to in vivo implantation.

### Biocompatible Matrix Comprising β-TCP and Collagen

In some embodiments, a biocompatible matrix can comprise a β-TCP bone scaffolding material and a biocompatible collagen binder. β-TCP bone scaffolding materials suitable for combination with a collagen binder are consistent with those provided hereinabove.

A collagen binder, in some embodiments, can comprise any type of collagen, including Type I, Type II, and Type III collagens. In some embodiments, a collagen binder comprises a mixture of collagens, such as a mixture of Type I and Type II collagen. In other embodiments, a collagen binder is soluble under physiological conditions. Other types of collagen present in bone or musculoskeletal tissues may be employed. Recombinant, synthetic and naturally occurring forms of collagen may be used in the present invention.

A biocompatible matrix, according to some embodiments, can comprise a plurality of β-TCP particles adhered to one another with a collagen binder. β-TCP particles suitable for use with a collagen binder can comprise any of the β-TCP particles described herein. In some embodiments, β-TCP particles suitable for combination with a collagen binder have an average diameter ranging from about 1 μm to about 5 mm. In another embodiment, β-TCP particles suitable for combination with a collagen binder have an average diameter ranging from about 1um to about 1 mm, from about 1 mm to about 2 mm, from about 1 mm to about 3 mm, from about 250 μm to about 750 μm, from about 250 μm to about 1 mm, from about 250 μm to about 2 mm, from about 200 μm to about 1 mm, or from about 200 μm to about 3 mm. β-TCP particles, in other embodiments, have an average diameter ranging from about 100 μm to about 300 μm. In a further embodiment, β-TCP particles suitable for combination with a collagen binder have an average diameter ranging from about 75 μm to about 300 μm. In additional embodiments β-TCP particles suitable for combination with a collagen binder have an average diameter less than about 25 μm and, less than about 1 mm or less than about 1 μm. In some embodiments, β-TCP particles suitable for combination with a collagen binder have an average diameter ranging from about 100μm to about 5 mm or from about 100 μm to about 3 mm. β-TCP particles, in some embodiments, can be adhered to one another by the collagen binder so as to produce a biocompatible matrix having a porous structure. In some embodiments, a biocompatible matrix comprising β-TCP particles and a collagen binder can comprise pores having diameters ranging from about 1 μm to about 1 mm. A biocompatible matrix comprising β-TCP particles and a collagen binder can comprise macropores having diameters ranging from about 100 μm to about 1 mm, mesopores having diameters ranging from about 10 μm to 100 μm, and micropores having diameters less than about 10 μm.

A biocompatible matrix comprising β-TCP particles and a collagen binder can have a porosity greater than about 25% or greater than 40%. In another embodiment, the biocompatible matrix can have a porosity greater than about 50%, greater than about 60%, greater than about 65%, greater than about 70%, greater than about 80%, or greater than about 85%. In a further embodiment, the biocompatible matrix can have a porosity greater than about 90%. Porosity facilitates cell migration and infiltration into the matrix for subsequent bone formation.

A biocompatible matrix comprising β-TCP particles, in some embodiments, can comprise a collagen binder in an amount ranging from about 1 weight percent to about 70 weight percent, from about 5 weight percent to about 50 weight percent, from about 10 weight percent to about 40 weight percent, from about 15 weight percent to about 35 weight percent, or from about 15 weight percent to about 25 weight percent of the biocompatible matrix. In a further embodiment, a collagen binder can be present in an amount of about 20 weight percent of the biocompatible matrix.

A biocompatible matrix comprising β-TCP particles and a collagen binder, according to some embodiments, can be flowable, moldable, and/or extrudable. In such embodiments, the biocompatible matrix can be in the form of a paste or putty. A paste or putty can be molded into the desired implant shape or can be molded to the contours of the implantation site. In some embodiments, a biocompatible matrix in paste or putty form comprising β-TCP particles and a collagen binder can be injected into an implantation site with a syringe or cannula.

In some embodiments, a biocompatible matrix in paste or putty form comprising β-TCP particles and a collagen binder can retain a flowable form when implanted. In other embodiments, the paste or putty can harden subsequent to implantation, thereby reducing matrix flowability.

A biocompatible matrix comprising β-TCP particles and a collagen binder, in some embodiments, can be provided in a predetermined shape such as a block, sphere, or cylinder.

A biocompatible matrix comprising β-TCP particles and a collagen binder can be resorbable. In some embodiments, a biocompatible matrix comprising β-TCP particles and a collagen binder can be at least 30%, 40%, 50%, 60%, 70%, 75%, or 90% resorbed one year subsequent to in vivo implantation. In another embodiment, this matrix can be resorbed at least 5%, 10%, 20%, 30%, 40%, 50%, 60%, 70%, 75% or 90% within 1, 3, 6, 9, 12, or 18 months subsequent to in vivo implantation.

A solution comprising PDGF can be disposed in a biocompatible matrix to produce a composition for promoting bone fusion in spine fusion procedures according to embodiments of the present invention.

Incorporating PDGF Solution into a Biocompatible Matrix

The present invention provides methods for producing compositions for use in bone fusion procedures. In some embodiments, a method for producing a composition for promoting the fusion of bone comprises providing a solution comprising PDGF, providing a biocompatible matrix, and incorporating the solution in the biocompatible matrix. PDGF solutions and biocompatible matrices suitable for combination are consistent with those described hereinabove.

In some embodiments, a PDGF solution can be incorporated into a biocompatible matrix by soaking the biocompatible matrix in the PDGF solution. A PDGF solution, in another embodiment, can be incorporated in a biocompatible matrix by injecting the biocompatible matrix with the PDGF solution. In some embodiments, injecting a PDGF solution can comprise incorporating the PDGF solution in a syringe and expelling the PDGF solution into the biocompatible matrix to saturate the biocompatible matrix.

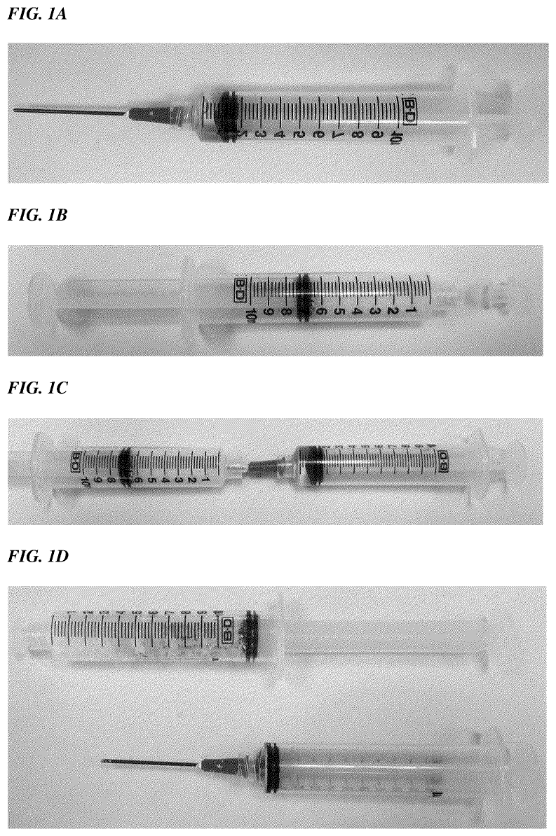

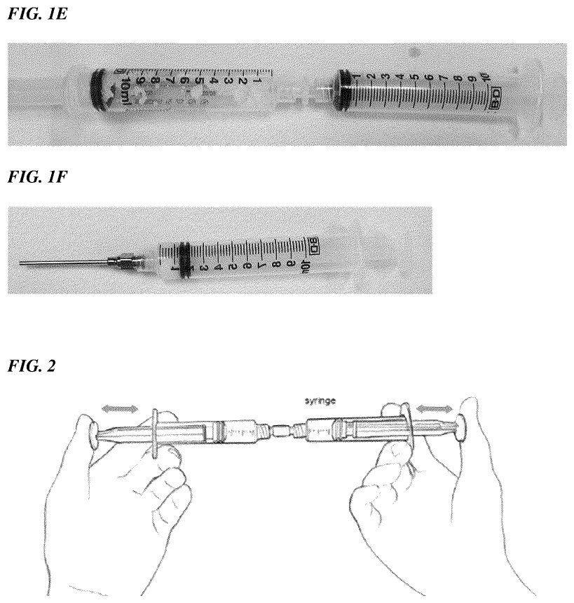

An embodiment of a method of incorporating solution into a biocompatible matrix is depicted in FIGS. 1A-1F. A solution of PDGF is withdrawn in to the barrel of a syringe (FIG. 1A). A second syringe containing the biocompatible matrix is also provided (FIG. 1B). The syringe containing the solution of PDGF is inserted into the syringe containing the biocompatible matrix and the PDGF solution is transferred into the syringe containing the biocompatible matrix to hydrate the biocompatible matrix (FIG. 1C). After transferring the PDGF solution, the hydrated biocompatible matrix may optionally be allowed to rest for a period of time, such as 1 to 10 minutes (FIG. 1D). The syringes are then connected to each other with, for example, a female-to-female lure lock connector, such that the are in fluid communication with one another (FIG. 1E). The biocompatible matrix and solution of PDGF are then mixed by transferring the hydrated biocompatible matrix back and forth between the syringes for several cycles (FIG. 2). In some embodiments, the contents are transferred for at least 5, 10, 20, or more cycles to form a homogenous paste. After mixing, the hydrated matrix may optionally be allowed to sit for several minutes prior to applying to a surgical site (FIG. 1F)

### Compositions Comprising Additional Biologically Active Agents

The compositions described herein for promoting and/or facilitating bone fusion in procedures, according to some embodiments, can further comprise one or more biologically active agents in addition to PDGF. Biologically active agents that can be incorporated into compositions of the present invention in addition to PDGF can comprise organic molecules, inorganic materials, proteins, peptides, nucleic acids (e.g., genes, gene fragments, small insert ribonucleic acids [si-RNAs], gene regulatory sequences, nuclear transcriptional factors, and antisense molecules), nucleoproteins, polysaccharides (e.g., heparin), glycoproteins, and lipoproteins. Non-limiting examples of biologically active compounds that can be incorporated into compositions of the present invention, including, e.g., anti-cancer agents, antibiotics, analgesics, anti-inflammatory agents, immunosuppressants, enzyme inhibitors, antihistamines, hormones, muscle relaxants, prostaglandins, trophic factors, osteoinductive proteins, growth factors, and vaccines, are disclosed in U.S. patent application Ser. No. 11/159,533 (Publication No: 20060084602). In some embodiments, biologically active compounds that can be incorporated into compositions of the present invention include osteoinductive factors such as insulin-like growth factors, fibroblast growth factors, or other PDGFs. In accordance with other embodiments, biologically active compounds that can be incorporated into compositions of the present invention preferably include osteoinductive and osteostimulatory factors such as bone morphogenetic proteins (BMPs), BMP mimetics, calcitonin, calcitonin mimetics, statins, statin derivatives, or parathyroid hormone. Preferred factors also include protease inhibitors, as well as osteoporotic treatments that decrease bone resorption including bisphosphonates, and antibodies to receptor activator of NF-KB ligand (RANK) ligand.

Standard protocols and regimens for delivery of additional biologically active agents are known in the art. Additional biologically active agents can be introduced into compositions of the present invention in amounts that allow delivery of an appropriate dosage of the agent to the implant site. In most cases, dosages are determined using guidelines known to practitioners and applicable to the particular agent in question. The amount of an additional biologically active agent to be included in a composition of the present invention can depend on such variables as the type and extent of the condition, the overall health status of the particular patient, the formulation of the biologically active agent, release kinetics, and the bioresorbability of the biocompatible matrix. Standard clinical trials may be used to optimize the dose and dosing frequency for any particular additional biologically active agent.

A composition for promoting bone fusion in spine fusion procedures, according to some embodiments, can further comprise the addition of other bone grafting materials with PDGF including autologous bone marrow, autologous platelet extracts, and synthetic bone matrix materials.

### Methods of Performing Fusion Procedures

The present invention also provides methods of performing bone fusion procedures, such as spine fusion procedures. In some embodiments, a method of performing a spine fusion procedure comprises providing a composition comprising a PDGF solution incorporated in a biocompatible matrix and applying the composition to a site of desired spine fusion. A composition comprising a PDGF solution incorporated in a biocompatible matrix, for example, can be packed into a site of desired spine fusion. In some embodiments, the composition can be packed such that the composition is in contact with the entire surface area of the bones in the bone fusion site. The composition may additionally be applied to the vicinity of the bone fusion site to further strengthen the fused bones.

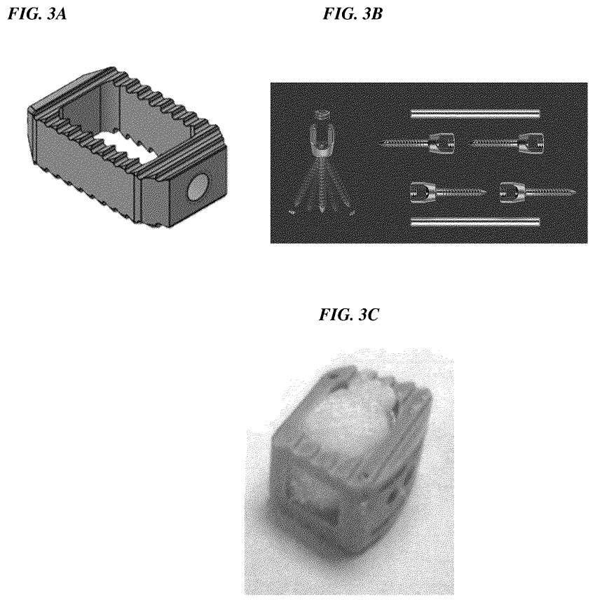

In some embodiments, the method comprises using a cage. The composition may be applied into the space inside the cage prior to insertion in the desired site of fusion, or alternatively, the cage may be placed at the desired site of fusion, and the composition is then applied to the space inside the cage. An exemplary cage is shown in FIG. 3A, and a cage filled with an exemplary composition of the present disclosure is shown in FIG. 3C. Additionally, fixation hardware also may be used to affix the cage at the site of fusion (FIG. 3B).

Vertebral bones in any portion of the spine may be fused using the compositions and methods of the present invention, including the cervical, thoracic, lumbar, and sacral regions.

In another embodiment, a method of the present invention comprises accelerating bony union in a spine fusion procedure wherein accelerating bony union comprises providing a composition comprising a PDGF solution disposed in a biocompatible matrix and applying the

The following examples will serve to further illustrate the present invention without, at the same time, however, constituting any limitation thereof. On the contrary, it is to be clearly understood that resort may be had to, various embodiments, modifications and equivalents thereof which, after reading the description herein, may suggest themselves to those skilled in the art without departing from the spirit of the invention.

### EXAMPLES

### Example 1

### Preparing a Composition Comprising rhPDGF-BB in a β-TCP/Collagen Matrix

A formulation of 0.3 mg/ml recombinant human platelet-derived growth factor (rhPDGF-BB) in 20 mM sodium acetate, pH 6.0 solution combined with a 20%/80% w/w bovine collagen/β-tricalcium phosphate matrix. When mixed at a 2:1 volume/mass ratio, the formulation results in a thick paste that can be applied through a 14G cannula or needle to the site requiring treatment.

The test formulation was prepared using 0.5 g of matrix (bovine collagen/β-TCP in a 20:80 w/w ratio) and 1 mL of a 0.3 mg/mL solution of rhPDGF-BB in 20 mM of sodium acetate, pH 6.0. A control article was similarly prepared with the 0.5 g matrix and 1.0 mL 20 mM of sodium acetate, pH 6.0. A 10 mL syringe equipped with an 18 gauge needle was used to draw 1.0 mL of the PDGF solution from a vial and into the barrel of the syringe (FIG. 1A). A second syringe containing 0.5 g of the β-TCP/collagen matrix was tapped several times in order to loosen the matrix and the syringe cap removed (FIG. 1B). The needle of the syringe containing the rhPDGF-BB solution was inserted into the needle hub of the syringe containing the β-TCP/collagen matrix and the rhPDGF-BB solution transferred into the syringe containing the matrix (FIG. 1C). While the rhPDGF solution is being transferred, the needle may be gradually moved out the needle hub of the matrix-containing syringe so that the rhPDGF solution hydrates as much of the matrix as possible. After transferring all of the rhPDGF-BB solution, the empty syringe was removed and the cap replaced on the needle of the syringe containing the hydrated β-TCP/collagen matrix (FIG. 1D). The hydrated matrix was allowed to sit undisturbed for at least two minutes. The empty syringe, which previously contained the rhPDGF solution, was connected to the syringe containing the hydrated matrix using a female-to-female luer lock connector (FIG. 1E). The hydrated matrix was then transferred back and forth between the two syringes for twenty cycles (a cycle being defined as passing the matrix into the empty syringe and then back into the original syringe) to form a homogenous paste. FIG. 2 is a schematic depicting the process of transferring the hydrated matrix between the two syringes in order to form the paste. The paste was finally transferred completely into one syringe. Any pressure built up during the mixing process may be relieved by gently pulling the plunger of the syringe. The empty syringe and female-to female luer lock connector were removed from the syringe containing the hydrated matrix. Any air remaining in the syringe was displaced and the a 14 gauge blunt cannula was attached (FIG. 1F).

### Example 2

### Evaluation of Lumbar Spine Fusion in an Ovine Model

### A. Study Design

An ovine model was chosen for this study because sheep have a spinal anatomy that is comparable in shape and length to humans. Additionally, sheep share similar bone healing processes to humans. Accordingly, an ovine model represents an acceptable translational model of lumbar spine fusion.

A total of 32 sheep underwent lumbar interbody fusion at the L2-L3 and L4-L5levels. Animals were divided into four treatment groups in which a PEEK interbody fusion cage was filled with autogenous bone graft (Group 1), rhPDGF-BB combined with collagen/β-TCP as described in Example 1 (Group 2), or collagen/β-TCP with sodium acetate vehicle (Group 3), or the PEEK cage was empty (Group 4). An exemplary PEEK spinal fusion cage is depicted in FIG. 3A, and fixation hardware is depicted in FIG. 3B. A cage filled with rhPDGF-BB combined with collagen/β-TCP as described in Example 1 is depicted in FIG. 3C. Treatment allocations were randomized between animals while fusion level was equally distributed for each treatment group. Animals were allowed to roam freely and eat ad libitum during the study and were sacrificed at either eight weeks or 16 weeks following surgery. Following sacrifice, spinal fusion quality was assessed through non-destructive kinematic biomechanical testing, micro-computed tomography and histology.

### B. Results

i. Biomechanics

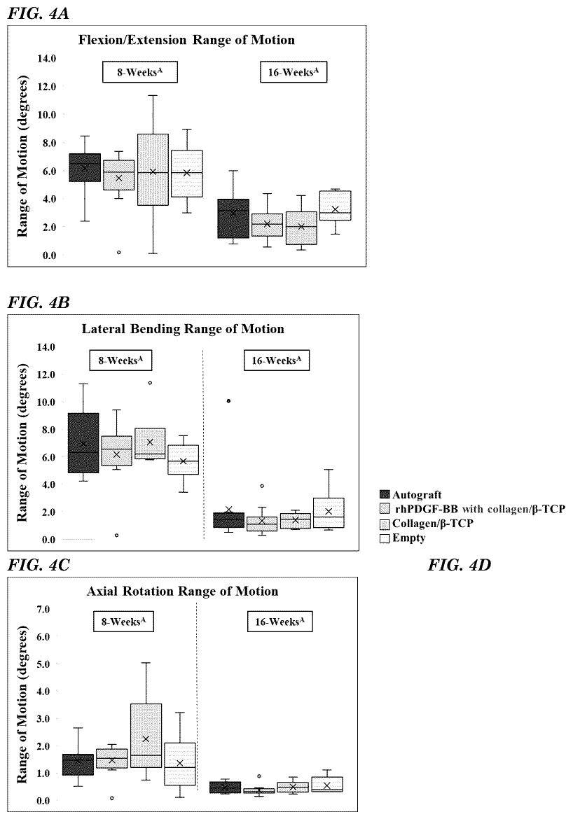

Non-destructive kinematic testing was performed on dissected L2-L3 and L4-L5functional spinal units under 6 Nm of pure moment loading in flexion-extension, left and right lateral bending, and left and right axial rotation. Overall, there was a statistically significant reduction in motion in all treatment groups in all principal directions from the 8-week to 16-week time point. There were no statistically significant differences between treatment groups within the 8-week or 16-week time point in any motion plane. The biomechanics results are depicted in FIGS. 4A-C (flexion extension, FIG. 4A; lateral bending, FIG. 4B; Axial rotation, FIG. 4C; key, FIG. 4D). Two-way Analysis of Variance (ANOVA) with α=0.05. Like-letters indicate statistically significant differences.
i. Micro CT

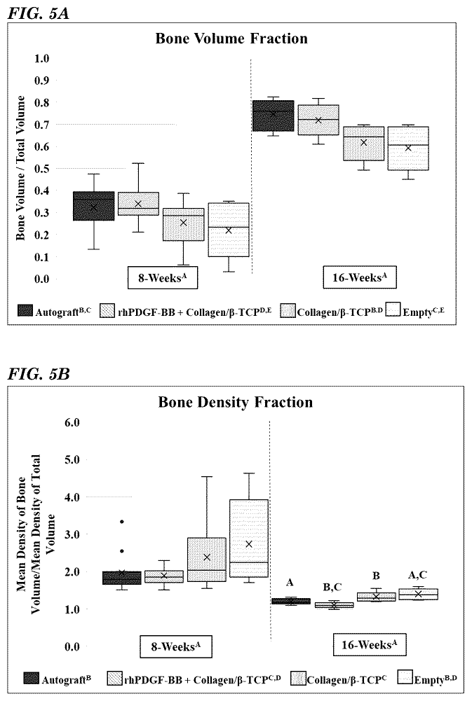

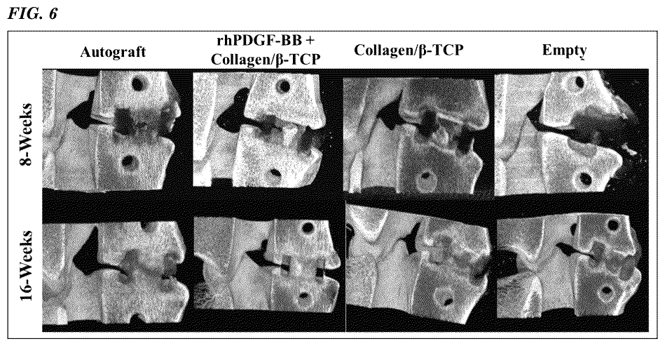

μCT analysis was performed on the core region of the cage. Bone volume and density were assessed within the PEEK interbody cage via MicroCT. There was a statistically significant increase in bone volume fraction within the interbody cage for all treatment groups from the 8-week to the 16-week time point (FIG. 5A). At the treatment level, collagen/β-TCP and empty treatments demonstrated significantly lower bone volume fraction as compared to autograft and rhPDGF-BB combined with collagen/β-TCP treatments; however, there was no statistically significant differences in bone volume fraction between treatment groups within either time point. (FIG. 5B). Two-way Analysis of Variance (ANOVA) with α=0.05. Like-letters indicate statistically significant differences.

Bone density fraction, a measurement of bone density normalized to the density of the entire analyzed volume, was quantified within each interbody cage. Greater bone consolidation is represented by numerical values closer to one. Again, there was a significant improved in bone density fraction for all treatment groups from the 8-week to 16-week time point. At the treatment level, rhPDGF-BB combined with collagen/β-TCP treatments resulted in statistically improved bone density fraction as compared to collagen/β-TCP and empty treatments. Further, at 16 weeks, animals treated with autograft bone demonstrated significantly improved bone density fraction as compared to animals treated with an empty PEEK cage while animals treated with rhPDGF-BB combined with collagen/β-TCP demonstrated significantly improved bone density fraction as compared to animals treated with either collagen/β-TCP alone or an empty PEEK cage.

Histological sections are typically taken in the sagittal plane through the interbody device to display the implant's core, anterior, and posterior surfaces, and the surrounding bones. Mid-sagittal cross-sections through the interbody cage demonstrate the typical bone formation for each treatment group at both time points, as seen in FIG. 6. Bone content was similar between autograft treated animals and animals treated with rhPDGF-BB with collagen/β-TCP at both time points. Less bone formation was seen in groups treated with collagen/β-TCP only or with no treatment.
iii. Histomorphometry

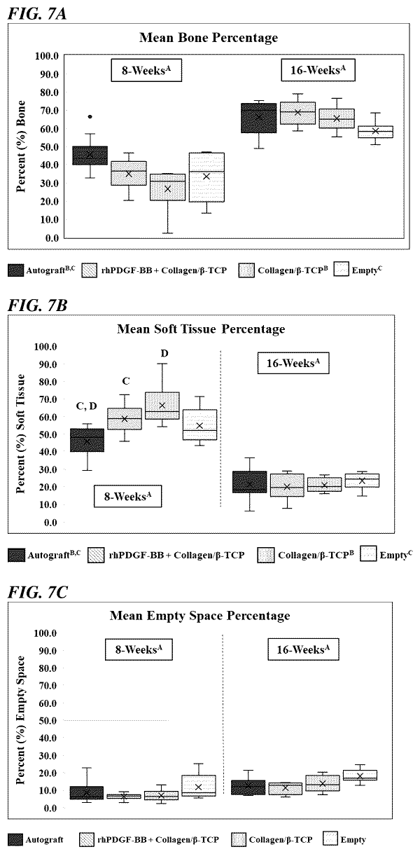

Histomorphometric evaluation of the fusion region bounded by the PEEK cage was performed to quantify the amount of bone, soft tissue, and empty area for each treatment. Mean bone percentage was significantly greater for all treatment groups at 16 weeks as compared to 8weeks. Animals treated with rhPDGF-BB combined with collagen/β-TCP did not demonstrate significantly different amounts of bone as compared to autograft treated animals at 8 weeks or 16weeks; however, animals treated with collagen/β-TCP alone resulted in significantly less bone as compared to autograft treated animals at 8 weeks. Mean soft tissue percentage was significantly lower for all treatment groups at 16 weeks as compared to 8 weeks. Animals treated with either rhPDGF-BB combined with collagen/β-TCP or collagen/β-TCP alone demonstrated significantly greater amounts of soft tissue as compared to autograft treated animals at 8 weeks. These differences were not present at the 16-week time point. Mean empty space within the interbody cage was significantly greater for all treatment groups at the later time point as compared to the early time point. No significant differences were observed between treatment groups within time points. At the treatment level, there were no statistically significant differences between autograft and rhPDGF-BB combined with collagen/β-TCP treatments in terms of bone, soft tissue, or empty space percentage. Results are summarized in FIGS. 7A-7C Two-way Analysis of Variance (ANOVA) with α=0.05. Like-letters indicate statistically significant differences. (mean bone percentage, FIG. 7A; mean soft tissue percentage, FIG. 7B; mean empty space percentage, FIG. 7C).

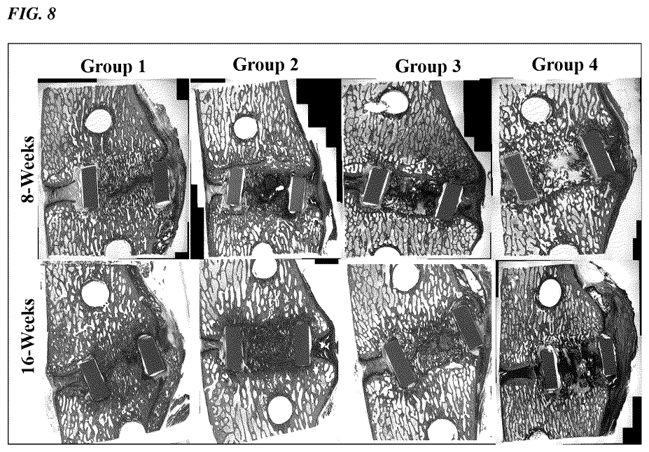

Histological sections are depicted in FIG. 8. These mid-sagittal cross-sections through the interbody cage again demonstrate the typical bone formation for each treatment group at both time points. The specimens are the same as the MicroCT representations shown in FIG. 6. Bone content was similar between autograft treated animals and animals treated with rhPDGF-BB with collagen/β-TCP at both time points. Less bone formation was seen in groups treated with collagen/β-TCP only or with no treatment.

### Example 3

### Evaluation of the Host Inflammatory Response to Rhpdgf-BB in a Pre-Clinical Rat Paraspinal Implant Safety Model

The neuroinflammatory host response of rhPDGF-BB when delivered in combination with a β-tricalcium phosphate (TCP)/collagen matrix carrier in vivo was evaluated in this example. Eighty Fischer F344 female rats underwent L4-5 posterolateral fusion with bilateral paraspinal muscle resection and graft placement using one of four implant types: 1) iliac crest “autograft” (allograft taken from syngeneic donors), 2) β-TCP/bovine collagen matrix (β-TCP/Col) with sodium acetate buffer, 3) β-TCP/Col with 0.3 mg of rhPDGF-BB, and 4) β-TCP/Col with 3.0 mg of rhPDGF-BB.

Animals underwent magnetic resonance imaging (MRI) and multiplex serum cytokine quantification at 4, 7, 10, and 21 days postoperatively. Spines and adjacent soft tissue were harvested and processed for histological evaluation staining with Ki67 & von Willebrand factor (vWF) to assess cell proliferation and neovascularization respectively, by immunofluorescence.

The cytokines evaluated in this study are listed in Table 1.

TABLE 1
List of serum cytokines included in analysis

| | | |
|---|---|---|
| EGF | IL-2 | IP-10 |
| Eotaxin | IL-4 | Leptin |
| Fractalkine | IL-5 | LIX |
| G-CSF | IL-6 | MCP-1 |
| GM-CSF | IL-10 | MIP-1α |
| GRO/KC/CINC-1 | IL-12p70 | MIP-2 |
| IFNγ | IL-13 | RANTES |
| IL-1α | IL-17A | TNFα |
| IL-1β | IL-18 | VEGF |

Four cytokines (GM-CSF, EGF, GRO/KC/CINC-1, and MIP-2) did not meet the minimum detectable concentration for any specimens and were excluded from further evaluation. Twenty of the remaining 23 serum cytokines analyzed by Luminex xMAP technology showed no significant differences among treatment groups when segregated by time point. Serum cytokine levels in the high-dose rhPDGF-BB and autograft groups are represented as fold change over time (FIG. 9). No clinically significant differences were found in serum levels of the other cytokines listed in Table 1 between control and rhPDGF-BB-treated animals.

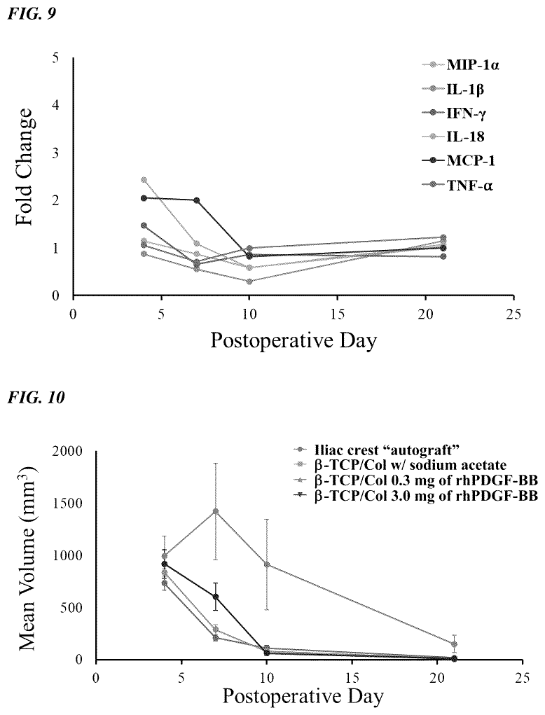

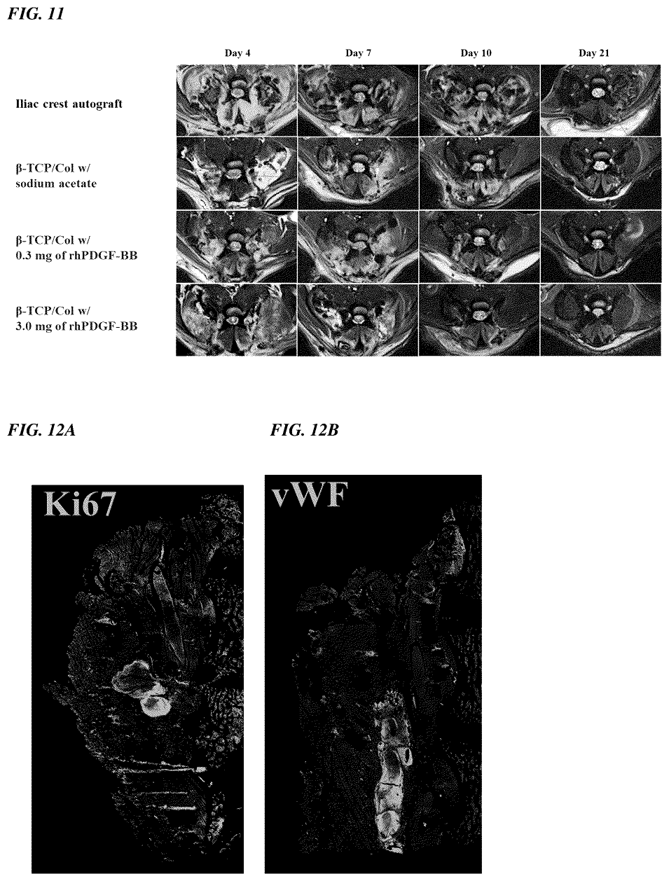

On MRI evaluation, there were no statistically significant differences in fluid accumulation among the treatment groups at any of the time points were found. The mean volumes of hyperintense areas on T2-weighted MRI images over time are depicted in FIG. 10. Images of epresentative T2-weighted axial MRI slices of the lumbar spine at L4-L5 disc space are shown in FIG. 11. Overlays representing the auto-segmented areas of hyperintensity indicated areas of inflammation.

FIGS. 12A and 12B are representative images of IF staining shown for Day 4 with β-TCP/Col 3.0 mg of rhPDGF-BB for Ki67 (FIG. 12A) and vWF (FIG. 12B). Slices were imaged with a TissueGnostic histological microscope (Zeiss) and were processed with the signal quantified using the software TissueFAXS (Zeiss)/NIS elements (Nikon). Quantified IF staining for Ki67 and von Willebrand Factor (vWF) shown at days 4, 7, 10 and 21 post-operatively.

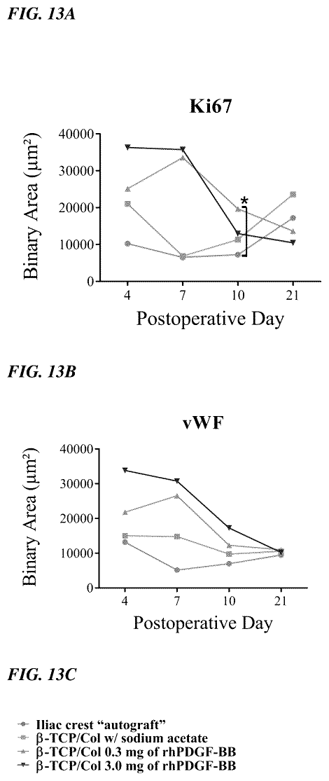

Quantified fluorescent signal of Ki67 (FIG. 13A) and vWF (FIG. 13B) were compared among groups at each time point using an ANOVA with post-hoc Tukey's test. There was a significant difference in Ki67 signal between iliac crest autograft and β-TCP/Col with 0.3 mg of rhPDGF-BB, at day 10 (p=0.013). There was no statistically significant difference between the other treatment groups.

In this pre-clinical paraspinal implant safety model, neither low-dose nor high-dose rhPDGF-BB induced a neuroinflammatory response in rats. Despite its role as a strong mitogenic and chemotactic agent for cells of mesenchymal origin, this study did not demonstrate any significant upregulation in inflammatory cytokines, increase in local tissue inflammation on MRI, or biologically significant difference in the angiogenesis associated markers that were evaluated by immunofluorescence.

All references disclosed herein are hereby incorporated by reference in their entirety.

Although there have been described particular embodiments of the present disclosure. it is not intended that such embodiments be construed as limitations upon the scope of this invention except as set forth in the following claims.

What is claimed is:

1. A composition comprising a solution of platelet-derived growth factor (PDGF) disposed in a biocompatible matrix, the biocompatible matrix comprising a bone scaffolding material and a biocompatible binder, wherein the solution has a PDGF concentration ranging from about 0.05 mg/mL to about 5 mg/mL, and wherein the solution and the biocompatible matrix are present in the composition in a volume to mass ratio (mL:g) ranging from about 1:1 to about 2.5:1.

2. The composition of claim 1, wherein the volume to mass ratio (mL/g) ranges from about 1.5:1 to about 2.5:1.

3. The composition of claim 1, wherein the volume to mass ratio (mL/g) is about 2:1.

4. The composition of any one of claims 1 to 3, wherein the scaffolding material is chosen from porous calcium phosphate, calcium sulfate, allograft and combinations thereof.

5. The composition of claim 4, wherein the porous calcium phosphate is selected from the group consisting of tricalcium phosphate, hydroxyapatite, poorly crystalline hydroxyapatite, amorphous calcium phosphate, calcium metaphosphate, dicalcium phosphate dihydrate, heptacalcium phosphate, calcium pyrophosphate dihydrate, calcium pyrophosphate, octacalcium phosphate and mixtures thereof.

6. The composition of claim 5, wherein the calcium phosphate comprises β-tricalcium phosphate.

7. The composition of any one of claims 1 to 6, wherein the bone scaffolding material comprises calcium phosphate and calcium sulfate.

8. The composition according to any one of claims 1 to 7, wherein the PDGF is present in the solution at a concentration ranging from about 0.1 mg/ml to about 1.0 mg/ml.

9. The composition according to claim 8, wherein the PDGF is present in the solution at a concentration from about 0.2 mg/ml to about 0.4 mg/ml.

10. The composition according to claim 8, wherein the PDGF is present in the solution at a concentration of about 0.3 mg/ml.

11. The composition according to any one of claims 1 to 10, wherein the PDGF comprises PDGF-AA, PDGF-BB, PDGF-AB, PDGF-CC, PDGF-DD, or a mixture or a derivative thereof.

12. The composition according to claim 10, wherein the PDGF comprises PDGF-BB.

13. The composition of any one of claims 1 to 12, wherein the PDGF consists of PDGF-BB.

14. The composition of claim 12 or 13, 68, wherein the PDGF-BB comprises at least 65% intact PDGF-BB.

15. The composition of any one of claims 12 to 13 wherein the PDGF-BB is recombinant human (rh) PDGF-BB.

16. The composition of any one of claims 1 to 15, wherein the solution comprises PDGF in a buffer.

17. The composition of any one of claims 1 to 15, wherein the solution consists of PDGF in a buffer.

18. The composition according to claim 16 or 17, wherein the buffer comprises sodium acetate.

19. The composition according to any one of claims 1 to 18, wherein the bone scaffolding material comprises particles in a range of about 50 microns to about 5000 microns in size, about 50 microns to about 5000 microns in size, about 100 microns to about 5000 microns in size, about 100 microns to about 5000 microns in size, about 100 microns to about 300 microns in size, about 100 microns to about 300 microns in size, or about 250 microns to about 1000 microns in size.

20. The composition according to any one of claims 1 to 19, wherein the bone scaffolding material comprises porosity greater than about 25%, greater than about 40%, greater than about 50%, greater than about 80% or greater than about 90%.

21. The composition according to any one of claims 1 to 20, wherein the bone scaffolding material comprises macroporosity.

22. The composition according to any one of claims 1 to 21, wherein the bone scaffolding material has a porosity that facilitates cell migration into the matrix.

23. The composition according to any one of claims 1 to 22, wherein the bone scaffolding material comprises interconnected pores.

24. The composition according to any one of claims 1 to 23, wherein the bone scaffolding material is resorbable.

25. The composition according any one of claims 1 to 24 the biocompatible binder comprises collagen.

26. The composition of any one of claims 1 to 25, wherein the biocompatible binder is present in the biocompatible matrix in an amount ranging from about 10 weight percent to about 40 weight percent.

27. The composition according claim 26, wherein the biocompatible binder is present in the biocompatible matrix in an amount ranging from about 15 weight percent to about 35 weight percent.

28. The composition according claim 26, wherein the biocompatible binder is present in the biocompatible matrix in an amount ranging from about 15 weight percent to about 25 weight percent.

29. The composition according claim 26, wherein the biocompatible binder is present in the biocompatible matrix in an amount of about 20 weight percent.

30. The composition according to any one of 1 to 29, wherein the biocompatible matrix consists of calcium phosphate and collagen.

31. A method for fusing bone comprising administering the composition of any one of claims 1 to 30 to a desired site of bone fusion.

32. The method of claim 31, wherein the site of bone fusion is a joint.

33. The method of claim 31 or 32, wherein the site of bone fusion is in a foot, toe, ankle, knee, hip, spine, rib, sternum, clavicle, joint, shoulder, scapula, elbow, wrist, hand or finger.

34. The method of claim 31 or 32, wherein bone fusion is in a spine fusion.

35. The method of claim 34, wherein the method further comprises placing an intravertebral spacer between vertebral bodies.

36. The method of claim 35, wherein the composition is disposed in the vertebral spacer prior to placing the vertebral spacer between the vertebral bodies.

37. The method of claim 35, further comprising disposing the composition in the spacer after placing the spacer between the vertebral bodies.

38. The method of any one of claims 34 to 37, wherein the spine fusion procedure is an interbody fusion procedure.

39. The method of any one of claims 34 to 37, wherein the spine fusion procedure is a lumbar fusion procedure.

40. A kit for use in a bone fusion procedure comprising:
a biocompatible matrix in a first container:
a solution of PDGF in a second container, wherein the biocompatible matrix and solution of PDGF have a volume to mass ratio (mL:g) ranging from about 1:1 to about 2.5:1, and
instructions for i) preparing a composition according to any one of claims 1 to 29, and ii) administering the composition to a site of bone fusion.
